(19) United States
(12) Patent Application Publication
(10) Pub. No.: US 2026/0124450 A1
(43) Pub. Date: May 7, 2026

(54) METHODS FOR TREATING BONE EROSION

(71) Applicant: SetPoint Medical Corporation, Valencia, CA (US)

(72) Inventors: Jacob A. LEVINE, West Hempstead, NY (US); David CHERNOFF, Sausalito, CA (US); Murthy V. SIMHAMBHATLA, Valencia, CA (US); Amy DEROSIER, Valencia, CA (US)

(21) Appl. No.: 19/378,237

(22) Filed: Nov. 3, 2025

Related U.S. Application Data
(60) Provisional application No. 63/715,497, filed on Nov. 1, 2024.

(51) Int. Cl. A61N 1/36; A61K 38/17; C07K 16/24

(52) U.S. Cl. CPC A61N 1/36053; A61K 38/177; A61N 1/36171; C07K 16/241

(57) ABSTRACT

Methods and apparatuses (e.g., devices and systems) for nerve stimulation to treat bone erosion, including in patients for having an inflammatory and/or autoimmune conditions, such as, but not limited to, rheumatoid arthritis (RA). In some cases, these methods may reduce or prevent bone erosion by applying electrical stimulation of the nerve (e.g., vagus nerve) in a patient that is desensitized to electrical nerve simulation to suppress inflammation (e.g., by decreasing cytokines such as RANK-L). In some cases this may include applying electrical energy in a manner that would desensitize the suppression of anti-inflammatory cytokines while reducing or preventing bone erosion. Alternatively or additionally, these methods may include applying electrical energy to a subject that is already desensitized to the electrical modulation of cytokines (e.g., RANKL) by nerve stimulation.

### CROSS REFERENCE TO RELATED APPLICATION

This patent application claims priority to U.S. Patent Application No. 63/715,497, filed on Nov. 1, 2024, titled “METHODS FOR TREATING BONE EROSION,” and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The disclosure relates generally to apparatuses (e.g., systems and devices) and methods of nerve stimulation to reduce bone erosion in patients regardless of treatment type, even in patients for which inflammatory therapies have failed.

### BACKGROUND

Bone erosion is a significant complication in the treatment and progression of osteoarthritis (OA), a degenerative joint disease marked by the breakdown of cartilage, joint inflammation, and the degradation of bone. It is currently believed that inflammatory processes, biomechanical stress, and metabolic changes all exacerbate bone erosion and complicate treatment. For example, in OA, inflammation is often localized in the synovium (the membrane that lines the joint) and produces pro-inflammatory cytokines, such as interleukin-1 (IL-1) and tumor necrosis factor-alpha (TNF-α). Current and proposed treatments for bone erosion typically modulate cytokines either directly or by treating synovitis, as it is believed that cytokines activate enzymes that break down cartilage and bone, leading to erosive damage, and that persistent inflammation accelerates bone loss by stimulating osteoclasts (bone-resorbing cells). However, treatment of synovitis and/or modulation of cytokine activity using anti-inflammatory drugs, including NSAIDs, may help reduce pain and inflammation but may not directly halt and may actually exacerbate bone erosion.

Previous work by the same inventors, including U.S. Pat. No. 10,449,358 (“the '358 patent”), taught that electrical stimulation of the vagus nerve with particular electrical stimulation parameters, in particular, extremely low duty cycle stimulation that was limited to a single session of less than about two minutes of stimulation every 12-48 hours at between 0.1 mA and 10 mA in amplitude would specifically reduce RANKL levels, increase OPG levels (improving the OPG/RANK ratio) specifically to inhibit osteoclast activity and bone resorption. Outside of these parameters, and in particular at higher duty cycle stimulation levels, the '358 patent taught that electrical stimulation would result in overstimulation of the vagus nerve and desensitization that would inhibit the reduction of bone erosion.

Although cytokines and other signaling molecules involved in osteoarthritis (OA) stimulate osteoclasts and modulate bone erosion, modulation of cytokines, including RANKL, to treat bone erosion may be an indirect treatment and may be challenging to titrate and may result in undesirable side effects. What is needed are direct and effective treatments for bone erosion to provide treatments.

### SUMMARY OF THE DISCLOSURE

The present invention relates to methods and apparatuses (systems and devices) for treating bone erosion by neurostimulation even in cases where neurostimulation is contraindicated or discontinued for some reason. In particular, the methods and apparatuses described herein may treat bone erosion by the application of electrical energy to a vagus nerve, a trigeminal nerve, a glossopharyngeal nerve, an optic nerve, and/or a facial nerve to reduce, eliminate or reverse bone erosion using simulation parameters that specifically reduce bone erosion independently of treating synovitis. For example, the methods and apparatuses described herein may apply electrical stimulation at levels previously believed to result in desensitization of the effect on cytokines, such as applying multiple session of electrical stimulation within a twelve hour period.

The methods and apparatuses described herein may be particularly effective at significantly reducing or preventing bone erosion which may be independent of the treatment of synovitis. This is significant because most approved drugs, and prior electrical treatments, to reduce bone erosion (particularly in osteoarthritis) treat bone erosion by reducing inflammatory synovitis which then indirectly reduces bone erosions.

The ability to specifically treat bone erosion more directly, rather than indirectly through treatment of synovitis, has many benefits. For example, these methods (and apparatuses for preforming them) may be used in patients that are experiencing undesirable bone erosion that is not necessarily due to inflammation and/or a change in RANKL (or OPG), such as a non-RANKL modulating bone erosion disorder. Further, these methods and apparatuses may specifically treat bone erosion using techniques that would be ineffective (or significantly less effective) for treatment of synovitis (and/or modulation of RANKL), which may also avoid unnecessary side effects.

As will be described in greater detail below, the methods and apparatuses described herein may apply electrical stimulation to a nerve (e.g., the vagus nerve) to inhibit bone erosion by modulation of osteoblasts and osteoclasts. Although the majority of the examples described herein are shown with respect to the vagus nerve, these methods and apparatuses may also be applied to one or more of: a trigeminal nerve, a glossopharyngeal nerve, an optic nerve, and/or a facial nerve. Specifically, stimulation of the never (e.g., vagus nerve) within the parameters described herein, e.g., may decrease bone resorption and increase bone synthesis. Thus, in general, these methods may include administering a nerve stimulation targeting one or more of: a vagus nerve, a trigeminal nerve, a glossopharyngeal nerve, an optic nerve, or a facial nerve to reduce, eliminate or reverse bone erosion. The stimulation parameters may be set as described herein.

Drugs for rheumatoid arthritis (RA) may modulate cytokines (and in particular TNF) including neutralizing circulating cytokines, in order to reduce inflammation and may help protect the joints of the patient taking the drugs, potentially reducing the progression of joint damage over time. Such therapies may also result in suppression inflammation. Similarly, patients treated with electrical stimulation of the vagus nerve (e.g., vagal stimulation) may also see a similar or even greater reduction (e.g., suppression) in inflammation and protection of joints. However, in some cases, it may not be desirable to suppress inflammation, or it may be desirable to limit the suppression inflammation.

When the bones of RA patients receiving this treatment were examined under MRI and scored to identify bone erosion, a dramatic reduction in bone erosion was seen over the first 12 weeks, and this effect continued for at least another 12 weeks. This effect was particularly robust a in subgroups including patients considered to have a very “erosive” phenotype, even in patents for whom drugs (such as biologic disease-modifying antirheumatic drugs, bDMARDs) had failed (e.g., lost efficacy). Surprisingly, even with a less than complete reduction in pro-inflammatory cytokines, the use of electrical stimulation of the vagus nerve (or a part of the vagus nerve reflex), can avoid immunosuppression while still reducing bone loss in patients receiving the therapy.

This may be due in part to the Applicant's observation that bone erosion may be a different pathway than inflammation. For example, the use of vagus nerve (or associated/communicating nerves, such as the splenic nerve) may suppress erosion using a different pathway than the inflammatory reflex and may therefore be triggered in a different population of patients and may be controlled in a different manner, as described herein. The mechanism of action for suppressing the inflammatory reflex may be different but overlapping with the mechanism of action for reducing bone erosion. For example simulation of the nerve may be done at different parameters and may result in a reduction in osteoclast and osteoblast activity even without an inflammatory effect. The reduction in bone loss may be caused by modulating osteoclast and osteoblast activity, even without suppressing the inflammatory response.

Further, although it was previously thought that the reduction in bone loss required signaling to bone cells to control RANKL (e.g., likely mediated by AChR), combinations of cytokines, such as tumor necrosis factor (TNF) plus interleukin-6 (IL-6), induce RANKL-independent osteoclastogenesis. For example, TNF/IL-6-induced osteoclast formation may result in RANKL-independent bone erosion, which may be treated by the methods and apparatuses described herein, including in inflammatory arthritis. Described herein are methods and apparatuses for employing these methods that may reduce bone erosion without modulating RANKL, and in particular, without immunosuppression. Specifically, these methods and apparatuses may beneficially reduce bone erosion by stimulation of a nerve (e.g., the vagus nerve), by electrical stimulation even in patients for whom the electrical stimulation does not substantially modulate inflammation (e.g., as measured by cytokine level(s)). Thus, in some cases patients for whom a stimulator (e.g., a vagus nerve stimulator) has been implanted in order to reduce inflammation, e.g., to treat RA or other inflammatory disorder, but who have either become insensitive or intolerant of the electrical stimulation, or who have achieved a therapeutically significant response, may continue to be treated (in some cases at higher or lower intensity, frequency and total charge delivered) by the implant to reduce bone erosion. Treatment of bone erosion may continue even as the patient uses other (e.g., drugs, including bDMARDs) are used. In some cases the treatment may be alternated between immunosuppressive treatment (e.g., at lower frequency, such as with an off-time of >12 hours) and higher frequency (applying energy multiple times within 12 hours) to alternate between immunosuppressive treatment and bone-erosion treatments.

In some cases the methods and apparatuses described herein may be used to treat patients receiving stimulation preferentially in the morning (e.g., between 4 am and noon, between 5 am and 10 am, etc.).

As mentioned above, it is particularly surprising that stimulation of the vagus nerve may reduce or reverse bone loss in patients for whom no significant immunosuppression is seen, particularly in light of earlier work suggesting that immunosuppression is necessary and concurrent with prevention of bone loss.

In general, these methods and apparatuses may be used to treat patients in combination with one or more anti-inflammatory drug, including in particular with one or more anti-inflammatory drugs after simulation of a nerve (e.g., vagus nerve) to treat inflammation has become less effective or ineffective. Thus, described herein are methods of treating a patient for whom electrical stimulation of the vagus nerve has failed, or become ineffective and/or who are taking anti-inflammatory drugs, such as, but not limited to bDMARDs. In such cases the patient may continue stimulation of the nerve (e.g., vagus nerve) using an implanted device and continue to benefit from a reduction (or reversal) in bone loss while taking one or more anti-inflammatory drugs, or without taking any anti-inflammatory drugs.

In general, these methods as described herein may be used to treat indications not limited to bone erosion, including osteoclast hyperactivity, osteitis, etc.

The implants may be implanted specifically for the treatment of bone erosion. In some cases, any of these methods may be performed in a patient into whom a nerve stimulator has been implanted and which is initially being used to treat inflammation. In some patients the treatment by the implanted nerve stimulation may have reduced or stopped being effective for treating inflammation. These methods may allow repurposing of the implant to treat bone erosion, e.g., to reduce or eliminate (and in some cases reverse) bone loss. Thus, the patient may continue to receive stimulation from the implant using stimulation parameters that are distinct from those used to treat inflammation in the same patient, even after the treatment for inflammation has stopped or decreased. As mentioned, in some cases the treatment for inflammation may be alternated with treatments for bone erosion. The implant may be operated as the parameters that are ineffective for treating inflammation, but that remain effective in reducing, preventing and/or reversing bone erosion.

In some cases the patient may discontinue inflammatory treatment with the implant and may continue using the implant to specifically treat bone, e.g., by reducing the intensity of the stimulation. Thus, the apparatuses described herein may be configured to operate in an immunosuppressive mode and a bone erosion mode. The immunosuppressive mode may limit the dose to one dose of 2 minutes or less per dose at a duty cycle of less than one dose every 12 hours (e.g., between 12-48 hours, such as having an off time of between 12-48 hours). The bone erosion mode may be less sensitive to off time, and may permit (or in some cases, enforce) greater than 1 dose every 12 hours. In some cases doses that are ineffective for reducing inflammation may be used to reduce or prevent bone erosion.

The intensity of the stimulation used to treat bone erosion (e.g., the peak amplitude of the applied electrical stimulation) may be the same or different as the intensity of the stimulation used to inhibit inflammation. In some cases the intensity of the stimulation between treatments that inhibit inflammation and treatments that inhibit bone erosion may be decreased (e.g., 5% or more, by 10% or more, by 15% or more, by 20% or more, by 25% or more, by 30% or more, by 35% or more, by 40% or more, by 50% or more, by 60% or more, by 70% or more, by 80% or more, by 90% or more, etc.). Reducing the stimulation parameters is counterintuitive, particularly as earlier work (e.g., looking at the effect on RANKL suggested that the same pathway for inflammation and bone erosion were the same and therefore the stimulation parameters should be the same, including the intensity. However, the work descried herein suggests that even lower levels of neural stimulation may be effective and may increase patient comfort. Note that intensity (energy applied during a dose) and timing of the dose (e.g., number of doses applied over time) may be separately modulated to differentiate between treating bone erosion and treating inflammation.

In cases in which a patient has been implanted with a nerve stimulation device (e.g., vagus nerve stimulation device) that is less effective or no longer effective for treating inflammation (e.g., RA, etc.), is particularly significant that the devices in such patient's may be adapted to effectively treat bone erosion, as it allows an already-implanted patients to benefit from effective treatment of bone erosion even after completing treatment of inflammation, or after becoming desensitized to the treatment of inflammation. Thus, from a practical perspective, if electrical stimulation of a never (e.g., vagus nerve stimulation, VNS) is not effective (or is less effective or no longer effective), the use of nerve stimulation may be continued, including increasing the frequency of dosing and/or decreasing the intensity of dosing, while continuing to treat bone erosion, including electrical treatment of bone erosion in combination with one or more additional drugs (e.g., bDMARDs) to treat inflammation.

As described in detail below, data from the study described herein also suggests that nonresponding patients for inflammation, e.g., patients for whom an implanted neural stimulator (e.g., VNS) do not (or no longer) result in reduction in inflammation may still surprisingly be treated for a reduction, elimination and/or reversal of bone erosion. Thus, as described herein the use of neural stimulation (e.g., VNS) may directly impact bone cells by a cytokine-independent mechanism, as demonstrated by the data provided herein.

Patients who have failed to respond or have become intolerant to either or both a drug therapy (such as a TNF inhibitor) and/or vagus nerve stimulation to treat inflammation (or an inflammatory disorder) may respond to the use of vagus nerve stimulation to treat bone erosion. This is surprising because prior to this work it was thought that treatments that activate the neuroimmune anti-inflammatory pathway rely on the same biological pathway as the drug therapy and as the bone erosion pathway (e.g., RANLK and OPG pathway) and therefore would not work in patients for whom the drug and/or the nerve stimulation is ineffective or has lost effectiveness to treat inflammation.

For example, described herein are methods of reducing bone erosion in a patient that is intolerant to or has failed to respond to an anti-inflammatory treatment (e.g., TNF-alpha inhibition, VNS, etc.). These methods may include applying nerve stimulation to treat bone erosion wherein applying nerve stimulation includes applying vagus nerve stimulation. Stimulation may be applied while the patient is also receiving treatment by one or more other anti-inflammatory treatment. The nerve stimulation may include more frequent dosing using electrical parameters that are otherwise similar to or less than the stimulation parameters (e.g., current/voltage intensity, pulse frequency, etc.) applied to the nerve and may comprise administering the treatment regimen to one or more nerve targets comprises one or more of: a cranial nerve, a peripheral nerve, a spinal nerve, or a nerve ending within an organ. In some examples applying the nerve stimulation comprises administering the treatment regimen to one or more of: a vagus nerve, a trigeminal nerve, a glossopharyngeal nerve, an optic nerve, or a facial nerve.

In general, the methods described herein include administering energy to one or more nerve targets. Typically this energy is electrical energy. In some cases, the energy is one or more of: electrical energy, mechanical energy, and thermal energy. In some examples applying energy comprises applying energy directly through contact or proximity, transdermally applying energy, and/or percutaneously applying energy. In general, applying energy may comprise applying energy with energy steering.

The peripheral nerve target may comprise one or more of: a radial nerve, a median nerve, an ulnar nerve, a femoral nerve, a sciatic nerve, a tibial nerve, a splanchnic nerve, a phrenic nerve, a hepatic nerve, a renal nerve, a splenic nerve, a nerve of the external ear, a greater auricular nerve, a lesser occipital nerve, an auriculotemporal nerve, and an auricular branch of the vagus nerve. The spinal nerve targets may comprise one or more of: a sacral nerve, a cervical nerve, a thoracic nerve and a lumbar nerve.

As mentioned above, applying the nerve stimulation in the patient may include administering the treatment regimen to one or more nerve targets including nerve ending within an organ. The nerve ending within the organ may be a nerve ending of one or more of: the spleen, the liver, the lymph nodes, the stomach, the small bowel, the large intestine, the pancreas, and the thymus. For example, administering the treatment regimen within the patient's brain may comprise administering the treatment regimen to one or more of: the locus coeruleus, motor nucleus of the vagus nerve, nucleus ambiguus, nucleus basalis, hypothalamus, and basal forebrain.

A peripheral nerve target may comprise one or more of: a radial nerve, a median nerve, an ulnar nerve, a femoral nerve, a sciatic nerve, a tibial nerve, a splanchnic nerve, a phrenic nerve, a hepatic nerve, a renal nerve, a splenic nerve, a nerve of the external ear, a greater auricular nerve, a lesser occipital nerve, an auriculotemporal nerve, or an auricular branch of the vagus nerve. The spinal nerve target may comprise: a sacral nerve, a cervical nerve, a thoracic nerve, or a lumbar nerve. The nerve ending within the organ may comprise a nerve ending within one or more of: a spleen, a liver, a lymph node, a stomach, an intestine, a pancreas, or a thymus.

As mentioned, applying the nerve stimulation in the patient may include applying electrical energy. For example, applying electrical energy may include applying a plurality of electrical stimulations each having a current that may be at or below a comfort level; in some cases the intensity may be sub-threshold (e.g., below the level of detection). Any of these methods may include setting the stimulation intensity level to be the same as, or less than, the stimulation level already used to treat a patient for inflammation, e.g., by x % (5%, 10%, 15%, 20%, 25%, 30%, 35%, 40%, 45%, 50%, 55%, 60%, 65%, 70%, 75%, 80%, etc.) or less than the level of intensity of anti-inflammatory stimulation.

Applying electrical energy may comprise applying nerve stimulations of less than 50 mA (e.g., 50 mA or less, 45 mA or less, 40 mA or less, 35 mA or less, 30 mA or less, 25 mA or less, 20 mA or less, etc.). In some examples applying electrical energy may comprise applying nerve stimulations of 2 mA or less (e.g., between about 0.1 and 2 mA, between 0.2 and 2 mA, between about 0.25 and 2 mA, between 0.1 and 1.5 mA, between 0.1 and 1 mA, between 0.2 and 1.5 mA, between 0.2 and 1 mA, etc.). In some examples applying electrical energy comprises applying nerve stimulations ranging from 1 μA to 500 μA (e.g., from 1 μA to 100 μA, from 1 μA to less than 100 μA, f''rom 1 μA to 90 μA, from 1 μA to 80 μA, from 1 μA to 70 μA, from 1 μA to 60 μA, from 1 μA to 50 μA, etc., from 20 μA to 200 μA). Applying electrical energy may comprise applying nerve stimulations ranging from 1 μA to 3 mA (e.g., from 1 μA to 3 mA, from 200 μA and 3 mA, etc.). The pulses may have duration of between about 1 μsec and 400 μsec, between about 20 μsec and 400 μsec, between about 50 μsec and 400 μsec, between about 100 μsec and 400 μsec, between about 1 μsec and 300 μsec, between about 20 μsec and 300 μsec, between about 50 μsec and 300 μsec, etc. The duration of the dose may be, e.g., between about 1 sec. and 5 minutes, between about 1 sec and 3 min, between about 1 sec and 2 min, between about 10 sec and 5 minutes, between about 10 sec and 3 min, between about 10 sec and 2 min, between about 30 sec and 5 minutes, between about 30 sec and 3 min, between about 30 sec and 2 min, between about 50 sec and 5 minutes, between about 60 sec and 3 min, between about 60 sec and 2 min, etc. In any of these methods, two or more doses may be applied within 12 hours (e.g., 3 or more within 12 hours, 4 or more within 12 hours, etc.).

When treating bone erosion, during delivery of a dose, applying electrical energy may comprise applying a charge ranging from 0.2 nanocoulombs and 5 kilocoulomb (e.g., over 5 minutes or less, over 10 minutes or less, over 15 minute or less, over 20 minutes or less, over 30 minutes or less, over 40 minutes or less, over 45 minutes or less, over 1 hours or less, over 1.5 hours or less, over 2 hours or less, over 2.5 hours or less, over 3 hours or less, over 4 hours or less, etc.).

Because the treatment of bone erosion evokes a different pathway than the cytokine-dependent pathway for suppressing inflammation, in general, the treatment of bone erosion may be provided in a manner that would desensitize the suppression of inflammation by electrical stimulation which has been previously described. For example, it was previously suggested that the RANKL-inhibition specific reduction in bone erosion (described, e.g., in U.S. Pat. No. 9,572,983) required the application of a dose of electrical energy at extremely low duty cycles, requiring a long rest period between doses (e.g., typically a 12-48 hour off time), in order to prevent desensitization. In contrast, the methods and apparatuses described herein suggest that such desensitization is not a factor. Thus, the methods and apparatuses described herein may be configured to apply more than one dose within the 12 to 48 hour period, such as applying two or more doses every 12 hours, three or more doses every 12 hours, 4 or more doses every 12 hours, etc.

In general, a dose may the application of energy at the treatment frequency for between 1 second and 10 minutes (e.g., between 10 seconds and 5 minutes, between 15 seconds and 4minutes, between 1 second and 3 minutes, between 1 second and 2 minutes, less than 5 minute, less than 4 minutes, less than 3 minutes, less than 2 minutes, and/or greater than 1 second, 2 seconds, 5 seconds, 10 seconds, 15 seconds, 20 seconds, etc.).

Although the examples provided here use electrical energy, these methods and apparatuses may be used with other energy application apparatuses, including applying mechanical energy (e.g., vibration, including ultrasound). Applying mechanical energy may comprise applying focused ultrasound within a frequency range 0.1-20 MHz. Applying mechanical energy may comprise applying focused ultrasound within a power range 0.01-10 mW/mm². Applying mechanical energy may comprise applying focused ultrasound within a pressure range of 10-1000 kPa. Applying electromagnetic energy comprise applying electromagnetic induction within a range of 1-500 A/m.

Applying electrical energy may comprise applying electrical energy from an implanted device. The implanted device may contain a pulse generator and at least one lead that terminates with at least one electrode. The electrode may comprise one or more of: a cuff electrode, a thin film electrode, a temperature-dependent form fitting elastomer, and an in situ setting injected conductive material. The at least one electrode may comprise a linear array of electrodes or a nonlinear array of electrodes. The implanted device may be a leadless device comprising a pulse generator and at least one electrode integrated onto the implanted pulse generator. The implanted device may be a transcutaneously powered leaded devices with a receiver that receives, stores, and/or transforms energy delivered from outside the body. The receiver may receive an electrical transmission, including nearfield, midfield, and far field transmissions, at amplitudes between 0.1 μA-500 mA. In some examples, the receiver may receive focused ultrasound energy at frequencies between 0.1-40 MHz and power between 0.01-50 mW/mm2 and pressure between 10-1000 kPa. In any of these examples, the receiver may receive energy through electromagnetic induction between 0.1-500 A/m.

The transcutaneously powered leaded device may contain at least one lead that terminates with at least one electrode. In some examples the implanted device comprises a leaded device powered by harvested energy with a receiver that receives, stores, and/or transforms energy from within the body.

Applying electrical energy may include applying to one or more of the vagus nerve and the splenic nerve, or a nerve that drive activation of the vagus nerve or splenic nerve.

In any of these methods described above, the neurostimulation can be administered according to a nerve stimulation regimen that includes applying a number of nerve stimulations doses to the patient over a period of time. The nerve stimulation regimen may be administered before, during, or after administration of one or more doses of an anti-inflammatory drug(s). The nerve stimulation treatments described herein may be combined with administration of one or more drugs used to reduce inflammation. In some examples, the drug(s) include one or more immunosuppressant and/or inflammation inhibitory drugs. In some cases, immunosuppressant drug(s) may include one or more disease-modifying antirheumatic drugs (DMARDs). The DMARD(s) can include synthetic drug(s) and/or a biological drug(s) and/or targeted synthetic drugs. The DMARD(s) can be conventional (csDMARDs) and/or biological (bDMARDs) and/or targeted (tsDMARDs). The drug(s) may include one or more enzyme inhibitors (e.g., Janus kinase (JAK) inhibitors), recombinant proteins, vaccines, blood products, gene therapy drugs, antibodies (e.g., monoclonal antibodies), small molecule drugs, and/or cell therapy drugs. In some cases, the drug(s) may be a glucocorticoid, such as prednisone, dexamethasone, and/or hydrocortisone.

In some cases, a nerve stimulation regimen to treat bone erosion may be implemented to effectively treat a patient's symptoms after it has been determined that drug treatment and/or nerve stimulation has been unsuccessful. For example, a method of treating bone erosion in a patient may include determining that administration of a drug and/or nerve stimulation of the patient has failed to achieve a low disease activity state in the patient and administering a nerve stimulation treatment regimen to achieve the low disease activity state.

For example, a nerve stimulation prescription to treat bone erosion can be added to a concurrently taken biological or targeted synthetic therapy, or biological or targeted synthetic therapy can be added to an active nerve stimulation treatment prescription, regardless of prior drug exposure. In some examples, nerve stimulation therapy to treat bone erosion can be implemented as an add-on to a drug therapy to improve or extend the clinical efficacy of the drug therapy.

In some cases, a nerve stimulation to treat bone erosion may be implemented if a patient is determined not to have undergone treatment using a particular drug, or particular class of drugs. For example, a method of treating bone erosion can include determining that the patient has become de-sensitized to a particular drug and applying a nerve stimulation regimen to treat bone erosion in the patient.

The apparatuses (devices and systems) and methods of using them described herein may incorporate some or all of the features of microstimulators, nerve cuffs (“PODs”), chargers, and programmer/controllers described herein may be similar or identical to those described in International Patent Application Publication No. WO 2011/028763, titled “PRESCRIPTION PAD FOR TREATMENT OF INFLAMMATORY DISORDERS;” U.S. Patent Application Publication No. US-2010-0312320-A1, titled “NERVE CUFF WITH POCKET FOR LEADLESS STIMULATOR;” and U.S. Pat. No. 9,993,651, titled “NEURAL STIMULATION DEVICES AND SYSTEMS FOR TREATMENT OF CHRONIC INFLAMMATION,” each of which is herein incorporated by reference in its entirety.

Any of the methods and apparatuses described herein may involve (e.g., may be applied after failure or de-sensitizing of the patient to) vagus nerve stimulation (VNS) to reduce of inflammation through activation of the cholinergic anti-inflammatory pathway. Any portion of the vagus nerve may be stimulated to treat bone erosion. In some examples, particular benefit is found when the sub-diaphragmatic vagus nerve and/or the cervical vagus nerve is stimulated.

Treatment and devices for treating inflammation by vagus nerve stimulation has been described, for example, in U.S. Pat. Nos. 6,838,471, 8,914,114, 9,211,409, 6,610,713, 8,412,338, 8,996,116, 8,612,002, 9,162,064, 8,855,767, 8,886,339, 9,174,041, 8,788,034, 9,211,410, and 9,993,651 and in International Patent Publication Nos. WO2016183353A1 and WO2017127758A1, each of which is herein incorporated herein by reference in its entirety.

These and other aspects and advantages are described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically illustrates one example of the modulation of inflammation and immunosuppression by vagus nerve stimulation (e.g., innate immunity).

FIGS. 2A and 2B schematically illustrates a potential pathway for vagus-nerve mediated joint protection. FIG. 2B shows the effect of disruption of this pathway (e.g., by cutting the vagus nerve).

FIG. 3 illustrates the study schematic for the study described herein, looking at the effects of nerve stimulation on inflammation (including RA) and of bone loss.

FIG. 4 is a table summarizing the baseline characteristics for all patients in a human trial showing electrical stimulation of a nerve (e.g., vagus nerve) to treat osteoarthritis and bone erosion.

FIG. 5 is a table summarizing baseline characteristics for a subset of the patients in the table of FIG. 4 having an erosive phenotype.

FIGS. 6-7 are graphs showing the change in erosion from baseline (cumulative probability) at 12 weeks in all subjects (FIG. 6) and subjects taking 1 biologic disease-modifying antirheumatic drugs (bDMARD).

FIGS. 8A-8B are graphs showing the change in erosion from baseline (cumulative probability). FIG. 8A (which is the same as FIG. 6, shown to allow comparison with 8B) shows change in erosion at 12 weeks in all subjects. FIG. 8B shows the change in erosion from baseline at 12-24 weeks in all subjects.

FIGS. 9A-9B are graphs showing the change in erosion from baseline (cumulative probability). FIG. 9A (which is the same as FIG. 7, shown to allow comparison with 9B) shows change in erosion at 12 weeks in subjects taking 1 bDMARD. FIG. 8B shows the change in erosion from baseline at 12-24 weeks in subjects taking 1 bDMARD from 12-24 weeks.

FIGS. 10A-10B are graphs showing the change in erosion from baseline (cumulative probability). FIG. 10A (which is the same as FIG. 6, shown to allow comparison with 10B) shows change in erosion at 12 weeks in all subjects. FIG. 10B shows the change in erosion from baseline at 12 weeks in subjects within the Peterfy subgroup.

FIGS. 11A-11B are graphs showing the change in erosion from baseline (cumulative probability) in the Peterfy subgroup of patients. FIG. 11A shows change in erosion at 12 weeks in the Peterfy subgroup of subjects. FIG. 11B shows the change in erosion from baseline at 12-24 weeks in the Peterfy subgroup of subjects.

FIGS. 12A-12B show the proportion of patients with progressions in erosions (e.g., greater than 0.5 erosion change) in all subjects comparing control and treatment groups (FIG. 12A) and in subjects experiencing a failure in 1 bDMARD at week 12 with progressions in erosion, comparing control and treatment groups (FIG. 12B).

FIGS. 13A-13B show the proportion of Erosion Progressor subjects with progressions in erosions (e.g., greater than 0.5 erosion change) in all subjects, comparing control and treatment groups (FIG. 12A), and in Erosion Progressor subjects with progressions in erosion experiencing a failure in 1 bDMARD, comparing control and treatment groups (FIG. 12B) at week 12.

FIGS. 14A-14B are graphs showing the change in synovitis from baseline (cumulative probability) in all subjects (FIG. 14A) and in subjects experiencing a failure in 1 bDMARD (FIG. 14B) at 12 weeks.

FIGS. 15A-15B are graphs showing the change in synovitis from baseline (cumulative probability) in subjects experiencing a failure in 1 bDMARD at 12 weeks (FIG. 15A, which is the same as FIG. 14B) at 24 weeks (FIG. 15B).

FIGS. 16A-16B are bar graphs summarizing the effects of nerve stimulation on bone erosion and cartilage loss on patients having an erosive phenotype after three months, as described herein, showing a significant reduction in bone erosion as compared to control patients (FIG. 16A) without any significant change in cartilage loss (FIG. 16B) over the same time period.

FIGS. 16C-16D are bar graphs summarizing the effects of nerve stimulation on bone erosion and cartilage loss on patients having an erosive phenotype after three months, as described herein, showing a significant reduction in osteitis (FIG. 16C) but no significant change in synovitis (FIG. 16D) over the same time.

FIG. 17 is a bar graph showing the percentage of subjects with erosion progression (>0.5) at 3 months of treatment as described herein.

FIG. 18 schematically illustrates one example of a method as described herein.

FIG. 19A schematically illustrates an example of an apparatus for applying electrical stimulation to reduce or prevent bone erosion.

FIG. 19B schematically illustrates another example of an apparatus for applying electrical energy to reduce or prevent bone erosion in which a microstimulator is separated from the electrodes by a lead.

### DETAILED DESCRIPTION

Described herein are methods and apparatuses (e.g., devices and systems) for nerve stimulation to treat bone erosion, including in patients for which one or more inflammatory and autoimmune conditions, such as, but not limited to, rheumatoid arthritis (RA). In general, these methods may include applying nerve stimulation (e.g., stimulation of the vagus nerve, such as electoral vagus nerve stimulation) to reduce or prevent bone erosion. In particular, these methods may include applying electrical stimulation of the nerve (e.g., vagus nerve) in a patient that is desensitized to electrical nerve simulation to suppress inflammation and/or RANK-L in order to reduce or prevent bone erosion. In some cases this may include applying electrical energy in a manner (e.g., dosing regimen) that would desensitize the effect on cytokines (e.g., desensitizing suppression of anti-inflammatory cytokines) while still reducing or preventing bone erosion, such as applying two or more doses within 12-48 hours (e.g., within 12 hours, etc.). Alternatively or additionally, these methods may include applying electrical energy to a subject that is already insensitive or desensitized to the electrical modulation of cytokines by nerve stimulation (e.g., vagus nerve stimulation).

For example, described herein is nerve stimulation to treat bone erosion in a patient already undergoing treatment for an inflammatory disorder in which the patients is or has become intolerant to, or have failed to respond to, treatment of an inflammatory disorder. These patients may have failed to respond (above a clinical threshold) to a drug therapy (e.g., TNF-alpha inhibitor) and/or therapy including none or more of: etanercept, adalimumab, certolizumab pegol, infliximab, golimumab, and biosimilars thereof. Surprisingly, the methods and apparatuses described herein for treating bone erosion may be used to treat a patient believed to be resistant to nerve stimulation to treat inflammation (e.g., vagus nerve stimulation). Further, these methods and apparatuses may be used to continue nerve stimulation in order to treat bone erosion when the patient has been found to be non-responsive or less responsive to the nerve stimulation (e.g., having an attenuated responses to nerve stimulation); other anti-inflammatory treatments may be applied, including the application of one or more drugs (e.g., TNF-alpha inhibition pharmaceutical agents, etc.).

Prior work has demonstrated that vagus nerve stimulation may provide therapeutic treatment for both inflammation, including osteoarthritis, and bone erosion. Specifically it was found that stimulation of the vagus nerve when treating inflammation results in a decrease in RANKL (Receptor Activator for Nuclear Factor κ B Ligand, RANKL), osteoprotegerin (OPG) and/or RANKL/OPG ratio by modulation of the vagus nerve concurrent with the treatment of inflammation. See, e.g., U.S. Pat. Nos. 9,572,983 and 10,449,358. In this initial work, vagus nerve stimulation (VNS) was applied at a level that was shown to modulate inflammation and in so doing, also modulate RANLK and/or OPG, thereby decreasing bone erosion.

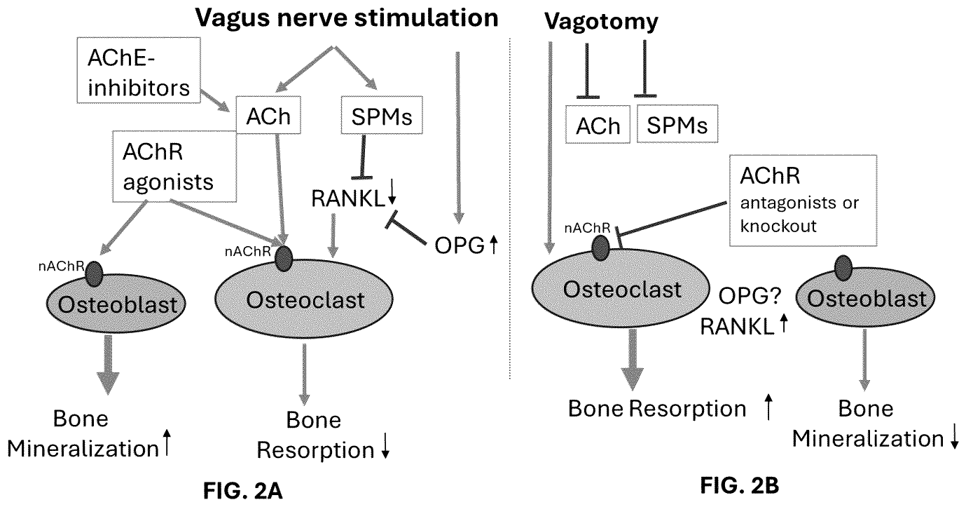

Surprisingly, the experiments described herein demonstrate a profound reduction in bone erosion in patients for whom vagus nerve stimulation does not significantly inhibit inflammation. In some cases the level of vagus nerve stimulation may set and/or maintained below the threshold for reducing inflammation, while still beneficially reducing bone erosion. This is particularly surprising because the biological pathways (as described in FIGS. 2A-2B) for reducing inflammation and for reducing bone erosion are believed to be similar, if not identical; it would reasonably be suspected that a therapy (e.g., neural stimulation) that is below the threshold for treating inflammation would also fail to treat bone erosion. However, as demonstrated by the data shown herein, this is not the case, suggesting that either a separate threshold is being activated and/or that different stimulation parameters may be used to treat bone erosion as compared with inflammation.

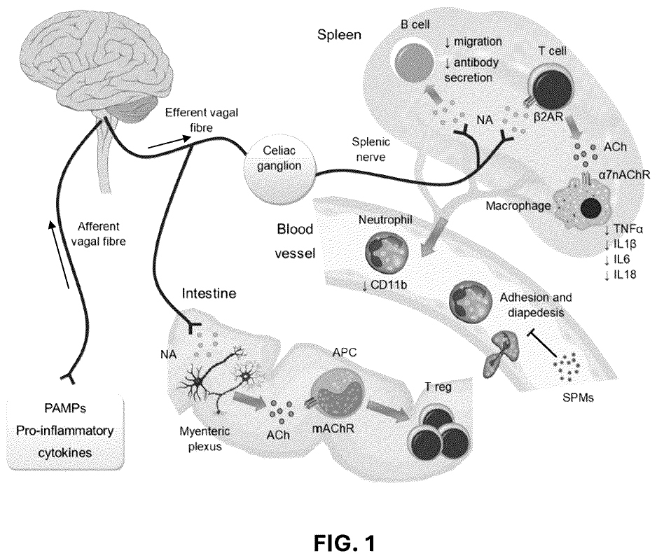

As illustrated in FIG. 1, the vagus nerve is known to modulate inflammation and actively coordinate immunoresolution. The this ‘inflammatory reflex’ may suppress a range of inflammatory cytokines by, e.g., between 30-70%; immunosuppressive biologics neutralize a single inflammatory pathway. The vagus nerve releases specialized pro-resolving mediators (SPMs) to aid resolution of inflammation by decreasing proinflammatory cytokines while increasing clearance of cellular debris. Stimulation, e.g., for 60 seconds, significantly reduces systemic TNF release; this suppression is sustained for 24-48 hours post stimulation.

Vagus nerve stimulation is also known to modulate joint protection, as schematically illustrated in FIGS. 2A-2B. As shown, Vagus nerve stimulation (VNS) may result in an increase in bone mineralization and a decrease in bone resorption, that is likely mediated through Acetylcholine receptors (AChR), as it can be inhibited by acetylcholine receptor agonists as well as by vagotomy (as shown in FIG. 2B). If this pathway is blocked, the bone is resorbed and bone mineralization is decreased.

Described here is a study of the effect of stimulation of nerves (e.g., the vagus nerves, and nerves that communicate with the vagus nerve, e.g., within the same pathway) on bone erosion. In this study, adults (22-75 years of age) with active moderate-to-severe rheumatoid arthritis (RA), despite ongoing treatment with conventional synthetic DMARD, who have had an inadequate response, loss of response or intolerance to at least 1 biologic or targeted synthetic DMARD (bDMARD) were enrolled in a two-part (two stage) trial. The first stage was weeks 0-12 and the second stage weeks 12-180. All patients remain on a stable background dose of at least 1 conventional synthetic DMARD through the primary endpoint evaluation (Week 12). Patients washed off biologic and targeted synthetic DMARDs prior to undergoing implant procedure and considered enrolled once implant is completed.

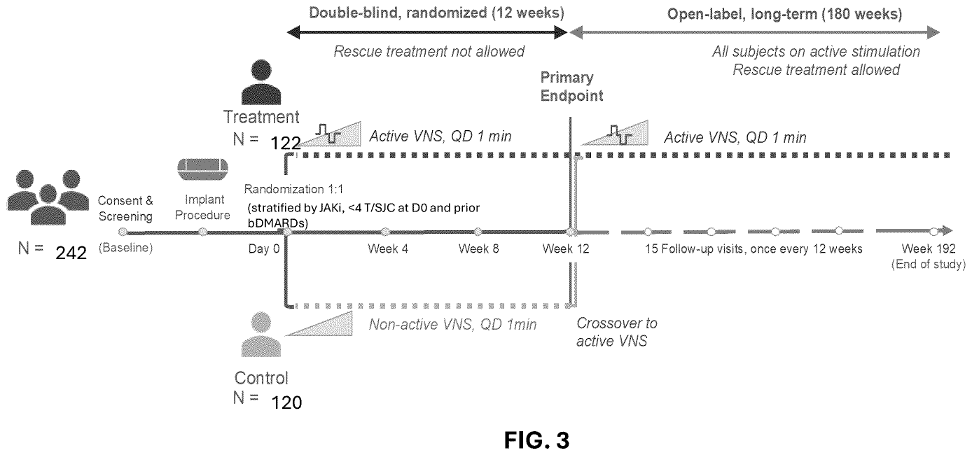

FIG. 3 shows an example of a schematic of the study, showing an operationally seamless, 2-stage, randomized, sham-controlled, double-blind, design having a 12-week follow-up, followed by one-way crossover of control group and an 180-week open-label follow-up. Enrollment included subjects meeting all the inclusion and none of the exclusion criteria were considered enrolled once implant procedure was attempted. The implant procedure including subjects meeting final eligibility will undergo placement of an implantable neurostimulator system in the neck, on the left cervical vagus nerve within the carotid sheath. The procedure took place in an operating room under general anesthesia and was performed by surgeons trained and experienced in procedures involving implantation of vagus nerve stimulators.

Within 14-21 days after implant procedure and after completing day 0 assessments, subjects were assigned randomly in a 1:1 ratio into either a treatment or control group using an interactive response technology. Randomization were stratified by prior Janus kinase inhibitors (JAKi) and RA severity at Day 0. Through Week 12, subjects assigned to the treatment group received active stimulation for 1 min once per day, and those assigned to the control group received non-active stimulation for 1 min once per day.

Primary end points at week Patients had the following options: receive active stimulation, receive rescue treatment and active stimulation, receive rescue treatment without active stimulation (may choose active stimulation later), or permanently turn off (decommission) implant. As follow-up, all patients were on active stimulation, and patients returned to the clinic every 12 weeks.

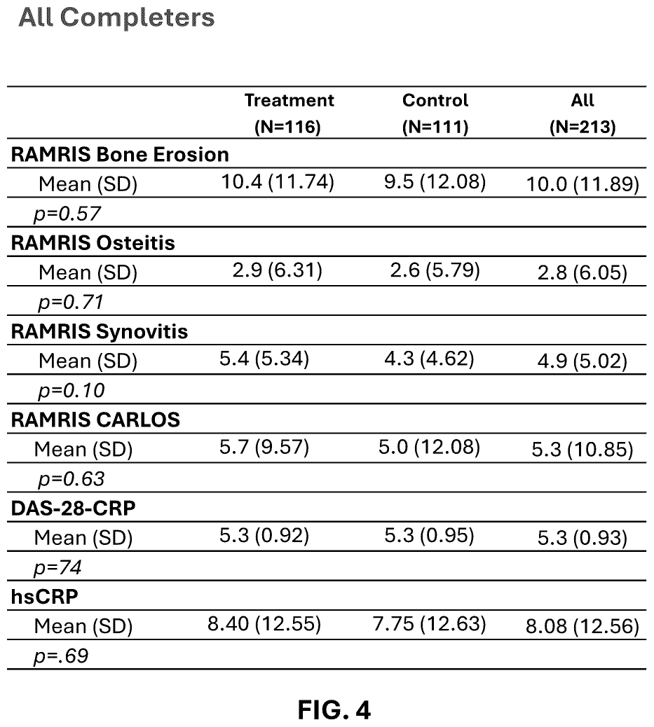

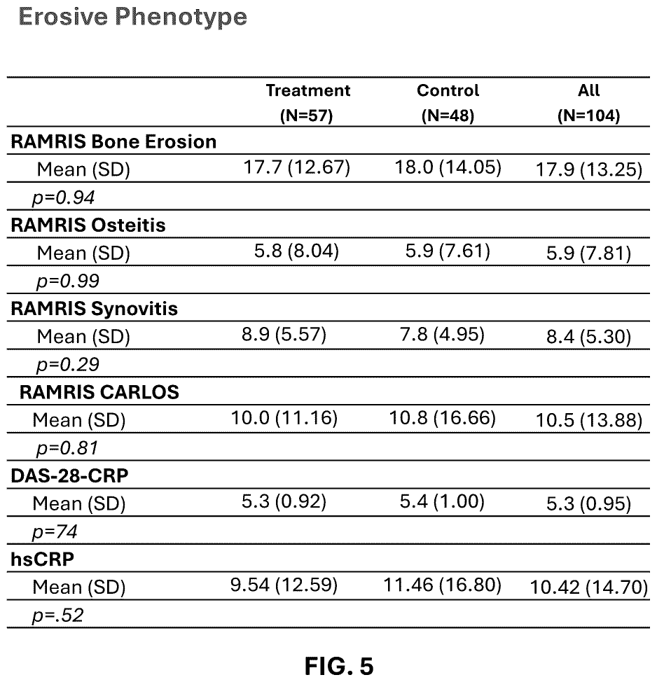

FIGS. 4-5 show tables illustrating the baseline characteristics for the patients in the study. The patent pool was divided up into all subjects within the trial (referred to herein as the “RAMRIS” trial), subjects having one bDMARD exposure, and subjects of erosive phenotype (Peterfy criteria). FIG. 4 shows characteristics (e.g., bone erosion, osteitis, synovitis, CARLOS, DAS-28-CRP and hsCRP) for all patients in the trial (e.g., treatment, control and all patients). The CARLOS score refers to a cartilage loss score, which is a semi-quantitative MRI-based scoring system used to evaluate cartilage loss in rheumatoid arthritis (RA). It was developed to complement the RAMRIS system, which primarily measures synovitis, osteitis, and bone erosion. RAMRIS stands for Rheumatoid Arthritis MRI Scoring system. It is an internationally accepted method to evaluate joint inflammation and damage in RA using MRI.

FIG. 5 shows baseline characteristics for the subset of patients in the study (from FIG. 4) having an erosive phenotype. These patients were identified as those at high risk for erosion progression, e.g., having one or more joint with a synovitis score of 2 or more (at baseline), or four or more joints with a synovitis score of 1 or more (at baseline), or one or more joint with osteitis (at baseline). Thus, a subgroup analysis was performed on patients with more severe baseline disease and at high risk for erosion progression, based on the presence of moderately severe synovitis, that is a RAMRIS-synovitis score of 2 or more, in at least one joint at baseline, or at least 4 joints with a synovitis score of 1 or 1 joint with active osteitis, as osteitis has been shown to be the strongest MRI predictor of bone erosion. Patients in this group constituted approximately half of the completer population and were referred to as the Erosive Phenotype, which is the focus of this report. Baseline values in the treatment and control arms for the Erosive phenotype were in good balance.

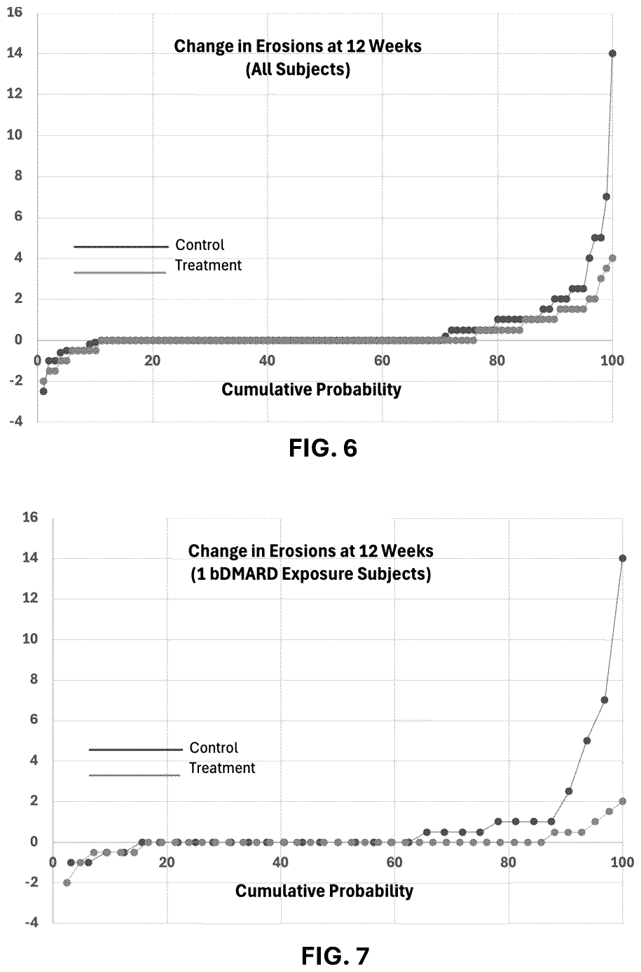

FIGS. 6-7, 8A-8B, 9A-9B, 10A-10B, 11A-11B, 12A-12B and 13A-13B illustrate changes is erosion in these different patient populations. For example, FIGS. 6-7 show the change in erosion from baseline (cumulative probability) at 12 weeks in all subjects, shown in FIG. 6, as compared to subjects taking 1 biologic disease-modifying antirheumatic drugs (bDMARD), shown in FIG. 7.

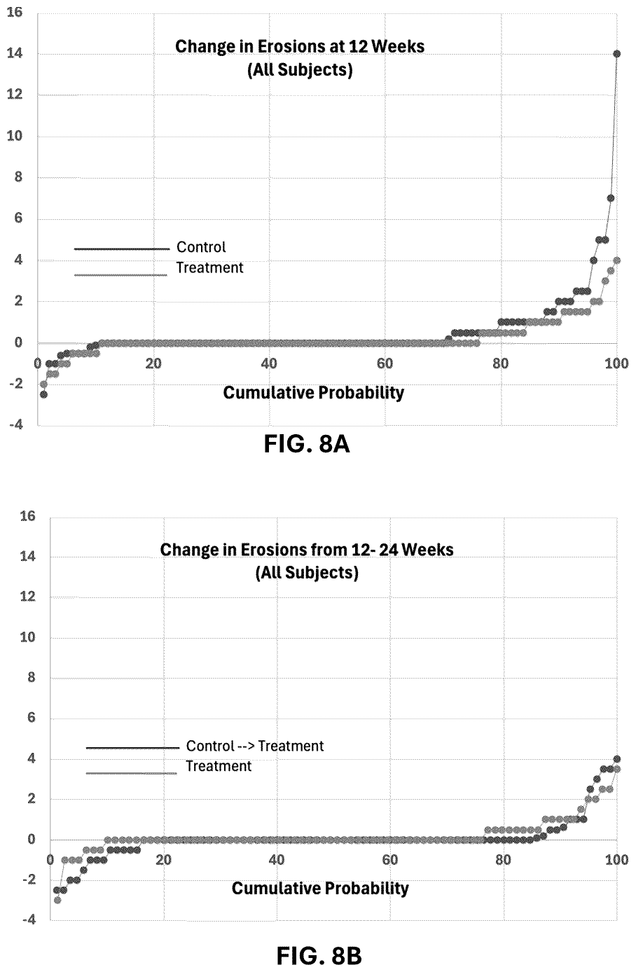

FIGS. 8A-8B show the change in erosion from baseline (cumulative probability). FIG. 8A (which is the same as FIG. 6) shows changes in erosion at 12 weeks in all subjects, while FIG. 8B shows the change in erosion from baseline at 12-24 weeks in these subjects. As mentioned, during the later part of the study (after week 12) the patients were permitted to apply or discontinue the treatment, including converting previous control patients to treatment patients, resulting in the flattening of the cumulative probability.

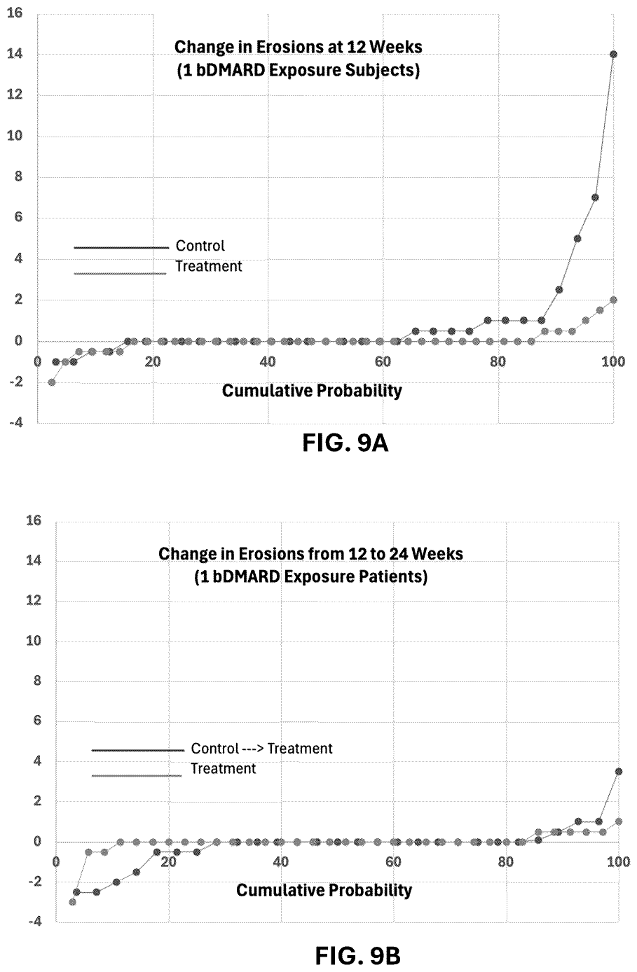

FIGS. 9A-9B show the change in erosion from baseline in which FIG. 9A (which is the same as FIG. 7, shown to allow comparison with FIG. 9B) shows change in erosion at 12 weeks in subjects taking 1 bDMARD. FIG. 8B shows the change in erosion from baseline at 12-24 weeks in subjects taking 1 bDMARD from 12-24 weeks. Similar trends are apparent.

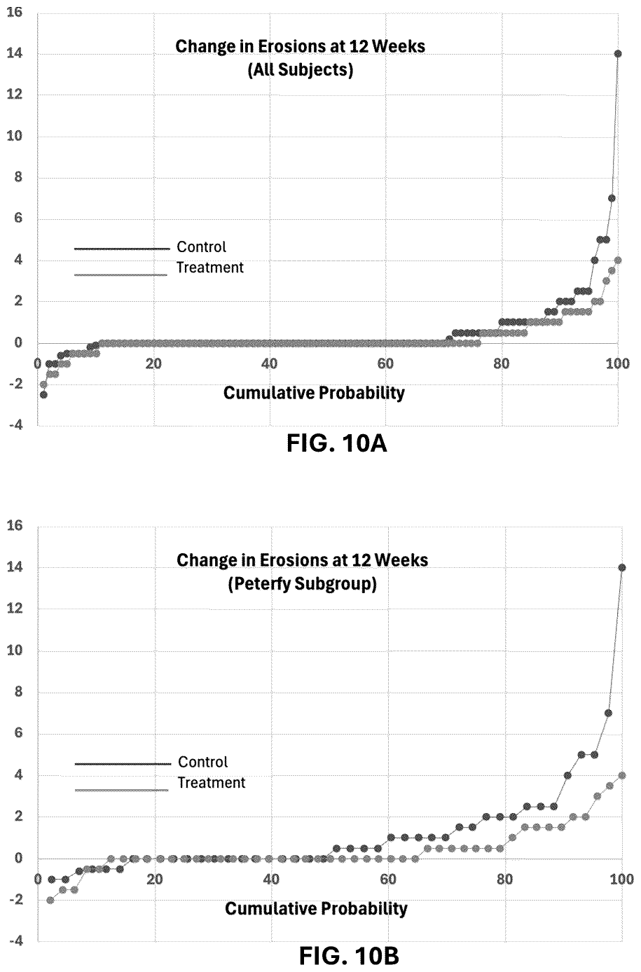

FIGS. 10A-10B show the change in erosion from baseline as a cumulative probability. FIG. 10A (which is the same as FIG. 6) shows change in erosion at 12 weeks in all subjects. FIG. 10B shows the change in erosion from baseline at 12 weeks in subjects within the Peterfy subgroup.

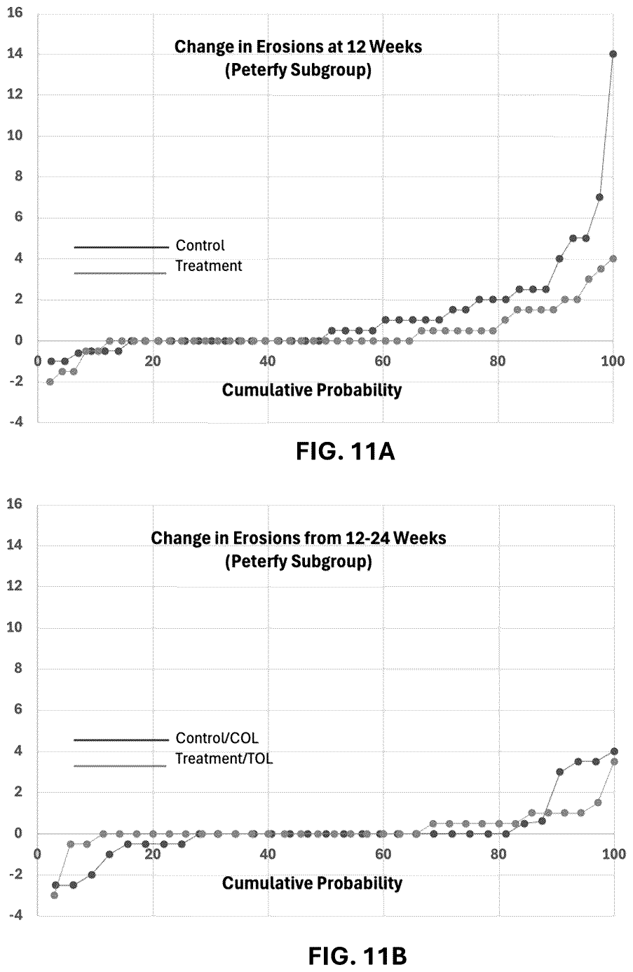

FIGS. 11A-11B show the change in erosion from baseline in the Peterfy subgroup of patients (e.g., patients having issues with bone erosion). FIG. 11A shows change in erosion at 12 weeks in the Peterfy subgroup of subjects. FIG. 11B shows the change in erosion from baseline at 12-24 weeks in the Peterfy subgroup of subjects.

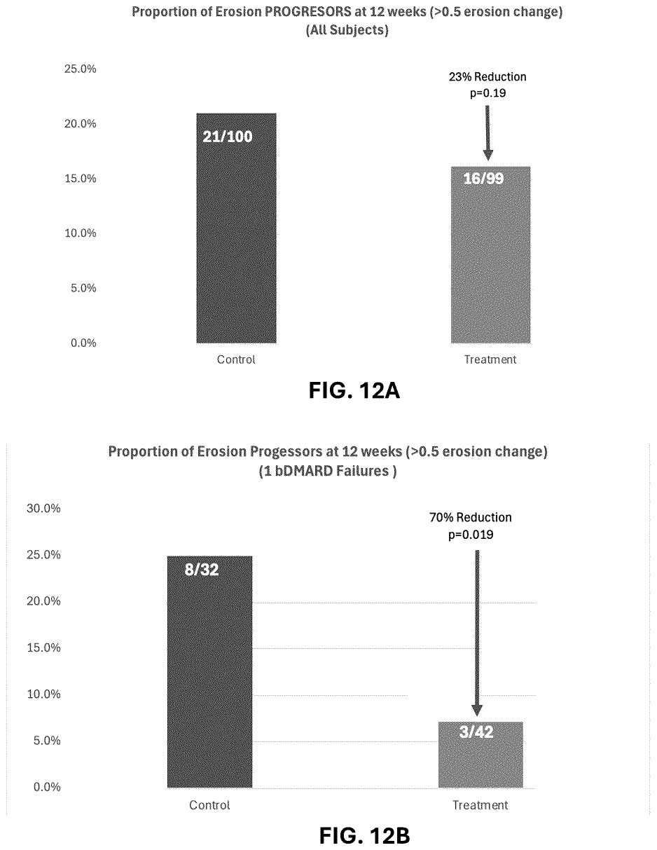

FIGS. 12A-12B show the proportion of patients with progressions in erosions (e.g., greater than 0.5 erosion change) in all subjects comparing control and treatment groups (FIG. 12A) and in subjects experiencing a failure in 1 bDMARD at week 12 with progressions in erosion, comparing control and treatment groups (FIG. 12B).

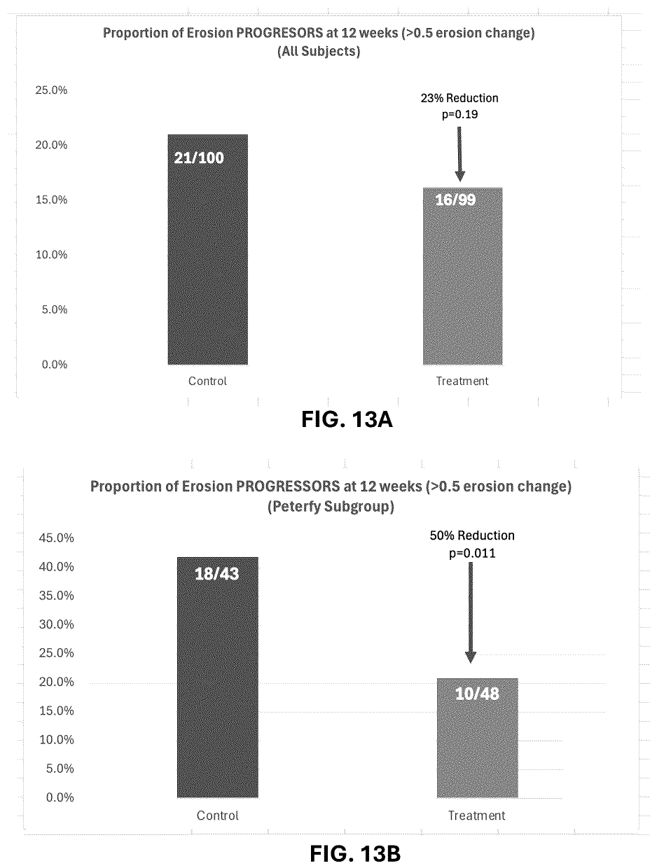

FIGS. 13A-13B show the proportion of Erosion Progressor subjects with progressions in erosions (e.g., greater than 0.5 erosion change) in all subjects, comparing control and treatment groups (FIG. 12A), and in Erosion Progressor subjects with progressions in erosion experiencing a failure in 1 bDMARD, comparing control and treatment groups (FIG. 12B) at week 12.

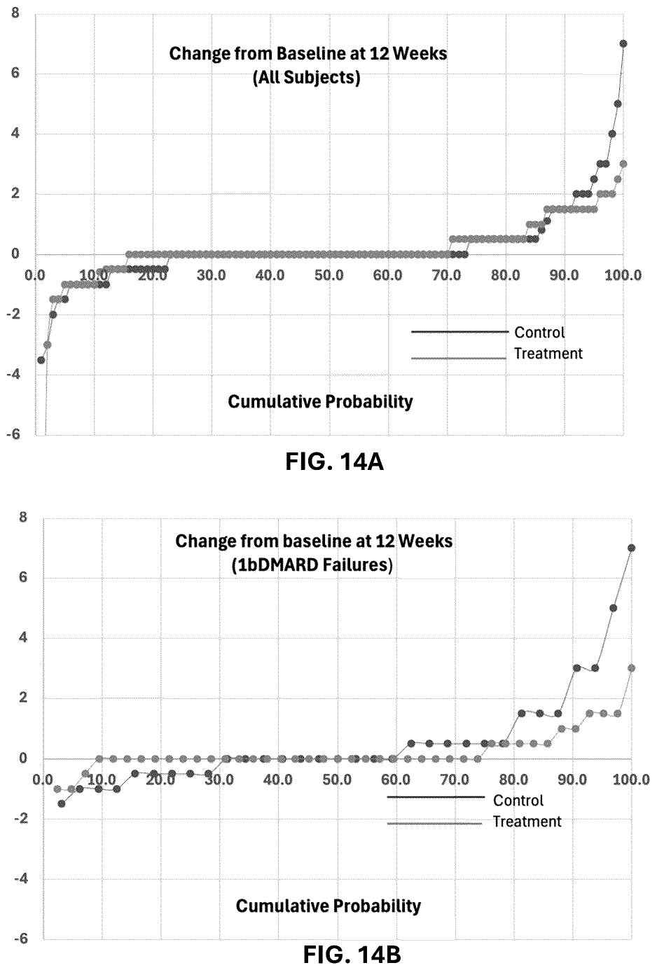

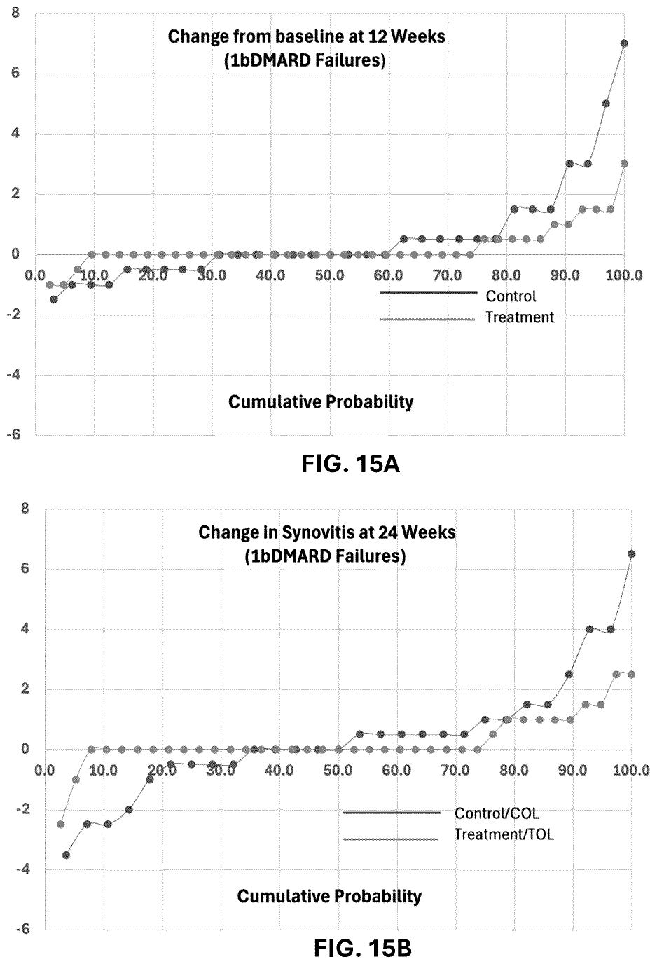

FIGS. 14A-14B and 15A-15B show data illustrating a change in synovitis for the same patient population. For example, FIGS. 14A-14B are graphs showing the change in synovitis from baseline (cumulative probability) in all subjects (FIG. 14A) and in subjects experiencing a failure in 1 bDMARD (FIG. 14B) at 12 weeks. Similarly, FIGS. 15A-15B show the change in synovitis from baseline (cumulative probability) in subjects experiencing a failure in 1 bDMARD at 12 weeks (FIG. 15A, which is the same as FIG. 14B) at 24 weeks (FIG. 15B).

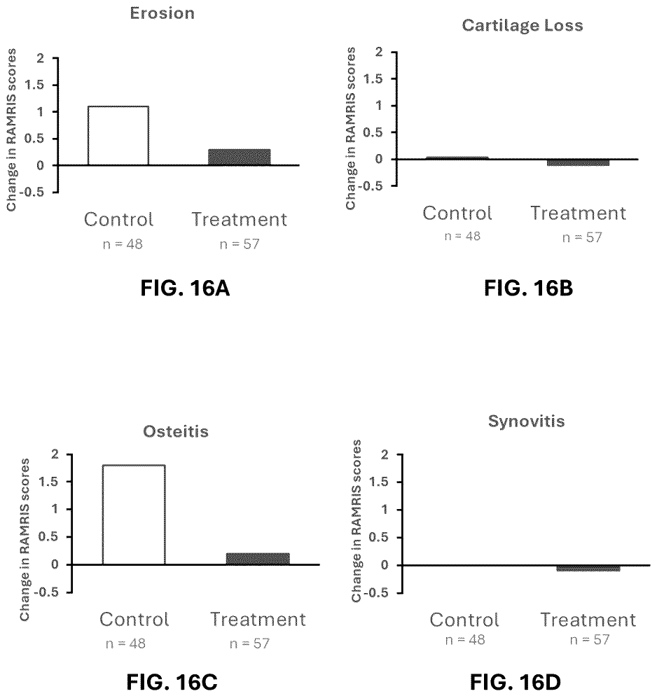

FIGS. 16A-16D illustrate the results of the MRI assessment of patients from the study. In general, these graphs show the changes in joint damage scores from baseline to 3 months, using MRI analysis. MRI is much more effective at detecting and monitoring change in joint damage. FIG. 16A shows the change in erosion (based on RAMRIS scores) between control patients and treatment patients. Patients in the Erosive Phenotype subgroup showed fairly aggressive progression in the control arm, with an increase in mean erosion score at 3 months that was slightly greater than that reported in most DMARD trials. Whereas patients in the treatment arm showed statistically significantly less progression in bone erosion than control, with an effect size similar to that seen in DMARD studies. At 6 months, both the treated subjects and the control subjects who crossed over to active treatment had essentially no progression in bone erosion, as shown in FIG. 16A. In contrast, little effect was seen on cartilage loss, as shown in FIG. 16B. Cartilage loss did not progress significantly in either arm of the study at 3 months or at 6 months, which is likely because cartilage loss typically presents only after bone erosion has gotten very severe.

FIG. 16C shows the change in osteitis. With regards to inflammation (e.g., hyperemia), patients in the control arm of the Erosive Phenotype subgroup showed worsening of osteitis whereas patients who received active stimulation had statistically significantly less increase at 3 months. At 6 months, both the treated subjects and the control subjects who crossed over to active treatment had decreased to baseline levels.

Surprisingly, particularly as compared with the effect on bone erosion, synovitis did not change from baseline for either group at 3 months or 6 months, as shown in FIG. 16D. Thus, bone erosion appears to be reduced independently of synovitis, which is surprising and not is no seen with drug therapies; for example, approved RA drugs typically work by reducing the inflammatory synovitis which then indirectly reduces bone erosions. The Applicants note that it is possible that the low score for synovitis may belie an actual change in inflammation, which may be difficult to detect with MRI. However, in such case, the methods described herein may still be used in patient's experiencing inflammation.

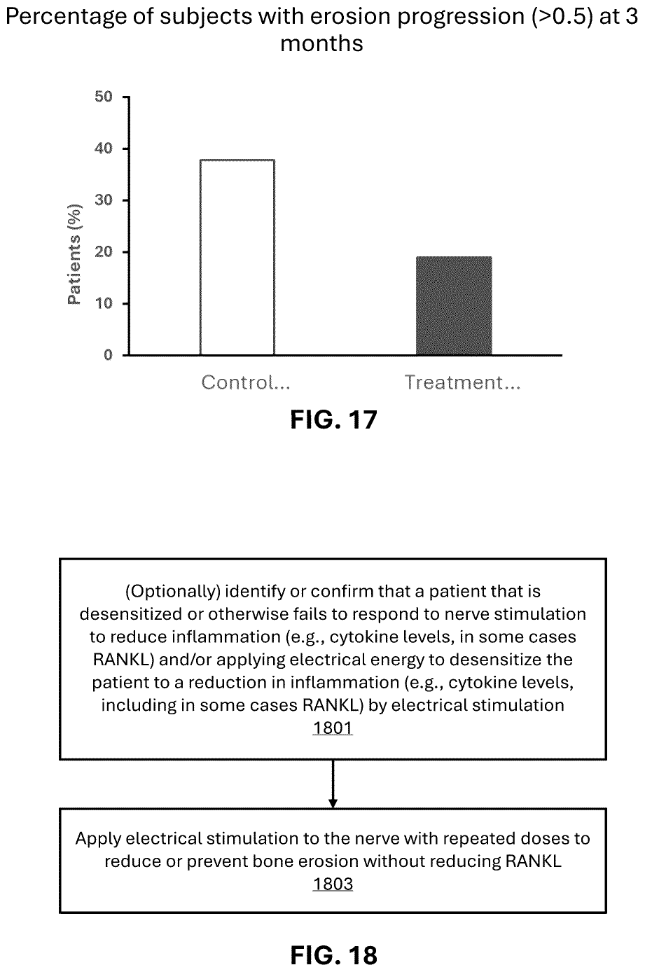

FIG. 17 summarizes the effect with respect to erosion progression. The percentage of patients in the Erosive Phenotype who showed any erosion progression on MRI was approximately 38% of the patients who were sham-treated progressed while only 19% of the patients in the actively treated arm progressed, a difference of about 50% which was a statistically significant.

### Methods

The methods and apparatuses (e.g., devices, systems, etc.) described herein may be used to treat a subject (e.g., a patient) to prevent or reduce bone erosion. These methods may include any of the features described herein.

For example, a method as described herein may be a method of reducing bone erosion in a patient that is desensitized to nerve stimulation to reduce a cytokine level, or that has failed to respond to nerve stimulation to reduce a cytokine level. In any of these methods the patient's status as desensitized to nerve stimulation to reduce cytokine levels may refer specifically to desensitized to nerve stimulation to reduce RANKL level. Any of these methods may include, as part of the steps of the method, confirming that the patient is desensitized to nerve stimulation to modulate cytokine (and particularly RANKL) level(s). This may be performed clinically, including by examining a patient having an implanted neurostimulator that is being treated to reduce inflammation and confirming inflammation and/or cytokine level(s) (e.g., RANKL level) is not reduced, or not further reduced, by treatment. For example blood cytokine levels may be examined as part of the method. Any of these methods may include driving stimulation at a level that will desensitize the patient to a reduction in inflammation (e.g., cytokine level, including RANKL), such as reducing the off-time of applied stimulation e.g., to less than, about 12 hours (e.g., by applying multiple doses of electrical stimulation as described herein within a 12 hour period).

As used herein, a subject that is desensitized to nerve stimulation to reduce a cytokine level, or that has failed to respond to nerve stimulation to reduce a cytokine level, may include a subject for whom the applied neural stimulation (e.g., vagus nerve stimulation) does not result in a change (e.g., reduction) in a cytokine level of greater than x %, where x is, e.g., 25%, 20%, 15%, 15%, 5%, etc. In some cases a subject (e.g., patient) that is desensitized to nerve stimulation to reduce a cytokine level, or that has failed to respond to nerve stimulation to reduce a cytokine level does not show a significant improvement in a measure (e.g., DAS28-CRP) of a cytokine or other marker (e.g., <15% change, <10% change, <5% change, etc.) after a minimum threshold of treatment (e.g., after 1 week, 1.5 weeks, 2 weeks, 3 weeks, 4 weeks, etc.).

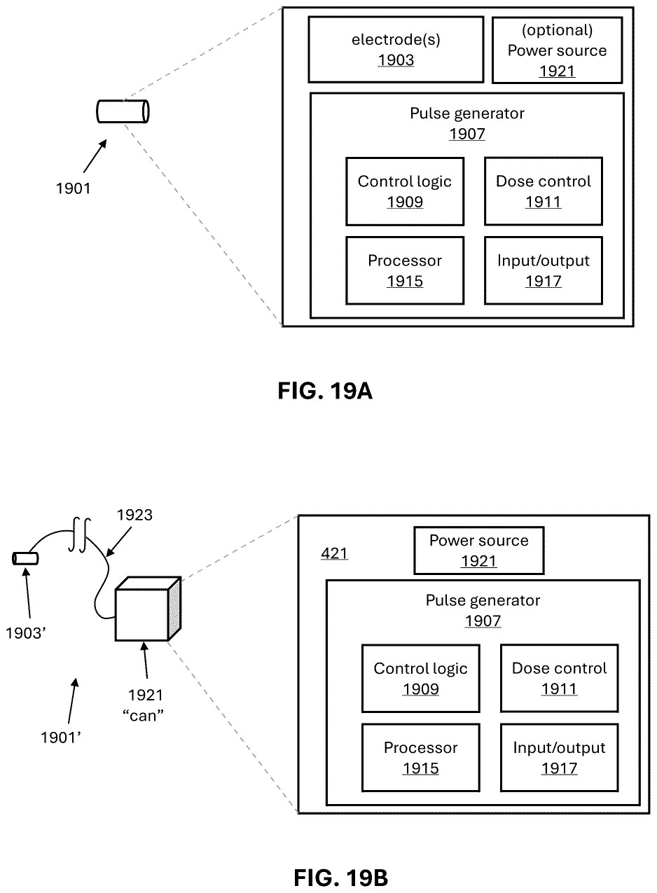

Any of these methods may include: applying electrical nerve stimulation to a nerve to reduce bone erosion, wherein the electrical stimulation is applied as a plurality of doses comprising a series of pulses of between 1 μA to 5 mA at a frequency of between 0.1 Hz and about 5 kHz, wherein each dose is between 1 second and 20 minutes in duration. In some cases the method may include applying electrical nerve stimulation from an implanted nerve stimulator (e.g., a microstimulator as described below in reference to FIGS. 19A-19B). Any appropriate nerve may be stimulated, including, the vagus nerve, and/or one or more of: a cranial nerve, a peripheral nerve, a spinal nerve, or a nerve ending within an organ. In some examples the nerve is the vagus nerve.

In general, any of these methods may include applying electrical nerve stimulation to reduce bone erosion by applying a plurality of doses within a 12 hour period.

Any of the methods described herein may include applying electrical nerve stimulation to the same nerve that was previously used to treat inflammation, e.g., using the same microstimulator (e.g., the same apparatus) on the same nerve (e.g., a vagus nerve). Thus any of these methods may include applying nerve stimulation at a first dose and set of stimulation parameters (e.g., a series of pulses of between about 1 μA to about 5 mA at a frequency of between about 0.1 Hz and about 5 kHz, wherein each dose is between about 1 second and about 20 minutes in duration) to reduce inflammation and/or reduce cytokines (including, in some examples, RANKL). The series of pulses may be between about 1 μA to about 1 mA. In some cases the series of pulses may have a frequency of between about 0.1 Hz and about 50 Hz.

The duration of each dose may be, for example, between about 1 second and about 2 minutes in duration.

FIG. 18 illustrates one example of a method of treating a patient to reduce or prevent bone erosion. In general, this method may optionally include identifying or confirming that a patient has been desensitized or otherwise fails to respond to nerve stimulation to reduce inflammation (e.g., cytokine levels, in some cases including RANKL), and/or applying electrical energy to desensitize the patient to reducing inflammation (e.g., cytokine, in some cases including RANKL) by electrical stimulation 1801 as described herein, for example, by applying doses less than 12 hours apart.

Any of these methods may then include applying electrical stimulation to the nerve with repeated doses to reduce or prevent bone erosion (e.g., without reducing RANKL) 1803.

For example, a method of reducing bone erosion in a patient that that is desensitized to nerve stimulation to reduce inflammation, or that has failed to respond to nerve stimulation to reduce inflammation, may include: administering a nerve stimulation using an implanted nerve stimulator, wherein the nerve stimulator is configured to treat inflammation by the application of a first set of stimulation parameters that have become ineffective or less effective to treat inflammation, further comprising applying nerve stimulation to the patient to treat bone erosion at these the first set of stimulation parameters or at a second set of stimulation parameters that are different from the first set of stimulation parameters.

The methods described herein may include methods of treating bone erosion in a patient, the method comprising: identifying that the patient has failed to respond to the application of electrical energy to a nerve of the anti-inflammatory pathway in the patient to reduce inflammation; and administering electrical energy to the nerve to treat bone erosion.

For example, administering electrical energy to the nerve to treat bone erosion may include applying the same intensity of electrical energy to the nerve as was applied to reduce inflammation. Administering electrical energy to the nerve to treat bone erosion may comprise applying an intensity of electrical energy to the nerve that is less than that applied to reduce inflammation. Administering electrical energy to the nerve to treat bone erosion may comprise reducing the off time between doses as compared to the application of electrical energy to a nerve of the anti-inflammatory pathway. The anti-inflammatory pathway in the patient may comprise the vagus nerve.

The anti-inflammatory pathway in the patient may comprise one or more nerve targets comprises one or more of: a cranial nerve, a peripheral nerve, a spinal nerve, or a nerve ending within an organ.

Any of these methods may include administering a drug agent to treat inflammation. The drug may comprise a TNF-alpha inhibitor comprising one or more of: etanercept, adalimumab, certolizumab pegol, infliximab, golimumab, and biosimilars thereof. In any of these methods the patient may have an immune or inflammatory disorder. For example, the patient may have an immune or inflammatory disorder that is one of: rheumatoid arthritis and psoriatic arthritis.

Any of these methods may include applying electrical energy comprises applying electrical energy from an implanted device. The implanted device may contain a pulse generator and at least one lead that terminates with at least one electrode. The electrode may comprise one or more of: a cuff electrode, a thin film electrode, a temperature-dependent form fitting elastomer, and an in situ setting injected conductive material.

A method of reducing bone erosion in a patient suffering from a non-inflammatory and/or non-RANKL modulating bone erosion disorder may include: administering a nerve stimulation targeting one or more of: a vagus nerve, a trigeminal nerve, a glossopharyngeal nerve, an optic nerve, or a facial nerve to reduce, eliminate or reverse bone erosion. The non-inflammatory bone erosion disorder may comprise one or more of: osteoporosis, osteoarthritis, gout, pseudo gout, and Rheumatoid arthritis, melorheostosis and mucopolysaccharidoses.

In any of these methods, nerve stimulation may target the vagus nerve. The nerve stimulation may be applied by an implanted nerve stimulator, including those described below in reference to FIGS. 19A-19B.

### Devices

Also described herein are apparatuses (e.g., devices, systems, etc.) for the treatment of bone erosion, as described above. These apparatuses may be implanted or external. In some cases external application of energy may be applied at the neck, e.g., at the level of the carotid and jugular. In some energy may be applied by implanting a nerve stimulator in communication with, e.g., the vagus nerve or a nerve that communicates with the vagus nerve. External simulation of the vagus nerve may be invasive (e.g., by injection or insertion of an electrode near the vagus nerve) or may be noninvasive (e.g., through one or more electrodes on the outer surface of the skin.

FIG. 19A shows an example of an implantable apparatus configured as an implantable microstimulator 1901 that may be configured to be inserted into a patient to treat the patient. In this example the apparatus 1901 may include hardware, software and/or firmware to control the application of energy to the nerve, as described herein, in order to treat bone erosion (or one or more disorders associated with bone erosion).

In FIG. 19A the microstimulator 1901 may include a pulse generator 1907 that is configured to generate and apply pulse energy as described by the parameters taught herein in order to treat bone erosion. In particular, the pulse generator 1907 may include one or more processors 1915, and a memory; the memory may store instructions (e.g., control logic) 1909 that may control operation of the apparatus to deliver electrical energy effective to treat bone erosion (e.g., to reduce or prevent bone erosion and/or to treat a disorder associated with bone erosion). Any of these pulse generators may include circuitry for controlling the receipt of energy from a power source or power storage 1921 component of the microstimulator and may generate a set of pulses having the dosing properties described herein. For example, the pulse generator may generate a series of pulses of between about 1 μA to 5 mA (e.g., between 1 μA and 4 mA, between 1 μA and 3 mA, between 1 μA and 2 mA, between 1 μA and 1 mA, between 1 μA and 500 μA, between 1 μA and 300 μA, between 1 μA and 200 μA, between 1 μA and 150 μA, between 1 μA and 100 μA, between 1 μA and less than 100 μA, etc.), at a frequency of between, e.g., about 0.1 Hz and about 5 kHz (e.g., between 1 Hz and 5 kHz, between 1 Hz and 3 kHz, between 1 Hz and 1 kHz, between 1 Hz and 100 Hz, between 1 Hz and 400 Hz, between 0.1 Hz and 10 Hz, etc.), and a pulse width of approximately (50-500 μsec, e.g., between 0.1 ms and 0.5 ms, between 0.2 ms and 0.5 ms, between 0.1 ms and 0.4 ms, between 0.2 ms and 0.4 ms, etc.) for a duration of between about 0.1 second and 10 minutes, e.g., between 0.1 second and 5min, between 1 second and 3 min, between 1 second and 2 min, between 1 second and 1 min, between 1 second and 50 seconds, between 1 second and 40 seconds, between 1 second and 30 seconds, between 1 second and 20 seconds, etc. The duration may refer to the duration of a single dose. As described herein, the apparatus may control the dose so that, for treating bone erosion, more than one dose may be applied within between about 12-48 hours (e.g., within about 12 hour, within less than 12 hours, etc.).

In particular, these apparatuses may include a dose control 1911 that causes a repeat of the dose within the minimum time period described above, e.g., two or more doses within every 12 hours. The control may be part of the pulse generator 1907 and may be integrated into the control logic 1909 or other components of the pulse generator.

The pulse generator 1907 may also include one or more inputs/outputs 1917 for communicating with one or more devices or systems outside of the implant. Any of these apparatuses may receive input and/or may provide output to a hand-held device (e.g., tablet, phone, etc.), including a dedicated device (e.g., external programmer/controller) that may be operated by the patient, doctor, technician, etc., and may allow programming, including uploading of programming, triggering the application of a dose (either manually and/or automatically), limiting the dose (e.g., to prevent dosing before a predetermined or adjustable “off time’ and/or causing a dose before a minimum time period, e.g., every 12 hours, etc.), and/or adjusting any of these dosing parameters.

In FIG. 19A, the microstimulator 1901 may include one or more (preferably 2 or more) integrated electrodes 1903 that may be placed in communication with the nerve (e.g., vagus nerve) once implanted. The electrodes may be on an outer surface of the implantable microstimulator 1901.

The microstimulator may include an optional power source 1921 that may be a battery, which may be rechargeable, by wireless recharging, e.g., inductive charging or the like, or the apparatus may be provided with on-demand power, e.g., configured to deliver power when energy is applied from an outside source (e.g., by an antenna). Any of these power sources 1921 may include power control circuitry.

FIG. 19B shows another example of an implantable microstimulator 1901′ that is not integrated (as in FIG. 19A), but that include the electrodes 1903′ separate from the microstimulator control circuitry (in the body of the microstimulator 1921) and is connected by a lead 1923. The body of the microstimulator may otherwise be similar or identical to that shown in FIG. 19A.

In any of apparatuses described herein, the controller may be configured to store two or more presets (“immunosuppressive” vs “bone-erosion”), which may be locked. In some cases these apparatuses may be configured so that the controller enforces a minimum off-time for one preset (e.g., immunosuppressive preset, for example, <one dose per 12 hours) and maximum off-time for the other (e.g., bone-erosion preset, for example, 2 or more doses per 12 hours). Any of these apparatuses may include a time-of-day scheduler (which may be configured to preferentially apply stimulation in the morning).

In use, any of these methods may include inserting/implanting the microstimulator into the body, e.g., implanting the electrodes within the cervical region of the neck in communication with the vagus nerve.

The methods and apparatuses described herein may be provided in combination with one or more bone-loss preventing drugs, and/or may be used when bone loss-preventing or erosion-reducing drugs become ineffective. For example, these methods and apparatuses may be used in combination with one or more drugs that slow down bone resorption (breakdown) by osteoclasts, helping to maintain or increase bone density, such as Bisphosphonates (e.g., Alendronate (Fosamax), Risedronate (Actonel, Atelvia), Ibandronate (Boniva), Zoledronic acid (Reclast)). These methods and apparatuses may be used in combination with one or more drugs that inhibit RANK Ligand (RANKL), such as Denosumab (Prolia, Xgeva). These methods and apparatuses may be used in combination with one or more drugs that mimic estrogen in the bone, helping to prevent bone loss, such as Selective Estrogen Receptor Modulators (SERMs), e.g., Raloxifene (Evista). These methods and apparatuses may be used in combination with one or more drugs such as Estrogen or estrogen-progestin therapy can help maintain bone density in postmenopausal women (e.g., Hormone Replacement Therapy, HRT, Estrogen Therapy, Estrogen-Progestin Therapy, etc. These methods and apparatuses may be used in combination with one or more drugs that stimulate new bone formation rather than just preventing bone loss, such as Parathyroid Hormone (PTH) Analogues (e.g., Teriparatide (Forteo), Abaloparatide (Tymlos)). These methods and apparatuses may be used in combination with one or more drugs such as sclerostin Inhibitors (e.g., Romosozumab (Evenity)), and/or Calcitonin (inhibits bone resorption), and/or Calcitonin (Miacalcin, Fortical), and/or Calcium and Vitamin D Supplements (e.g., Calcium Carbonate or Calcium Citrate, Vitamin D (D2 or D3).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and examples such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or examples of various examples. Combinations of the above examples, and other examples not specifically described herein, will be apparent to those of skill in the art upon reviewing the above description.

What is claimed is:

1. A method of reducing bone erosion in a patient that is desensitized to nerve stimulation to reduce a cytokine level, or that has failed to respond to nerve stimulation to reduce a cytokine level, the method comprising: applying electrical stimulation to a nerve to reduce bone erosion, wherein the electrical stimulation is applied as a plurality of doses comprising a series of pulses of between 1 μA to 5 mA at a frequency of between 0.1 Hz and 5 kHz, wherein each dose is between 1 second and 20 minutes in duration.

2. The method of claim 1, wherein applying electrical nerve stimulation comprises the electrical nerve stimulation from an implanted nerve stimulator.

3. The method of claim 1, wherein the nerve is the patient's vagus nerve.

4. The method of claim 1, wherein applying electrical nerve stimulation comprises applying a plurality of doses within a 12 hour period.

5. The method of claim 1, wherein applying electrical nerve stimulation comprises applying the nerve stimulation after applying nerve stimulation to treat inflammation to the same nerve.

6. The method of claim 1, wherein the series of pulses is between 1 μA to 1 mA.

7. The method of claim 1, wherein the series of pulses has a frequency of between 0.1 Hz and 50 Hz.

8. The method of claim 1, wherein each dose is between 1 second and 2 minutes in duration.

9. A method of reducing bone erosion in a patient that is desensitized to nerve stimulation to reduce inflammation, or that has failed to respond to nerve stimulation to reduce inflammation, the method comprising: administering a nerve stimulation using an implanted nerve stimulator, wherein the nerve stimulator is configured to treat inflammation by the application of a first set of stimulation parameters that have become ineffective or less effective to treat inflammation, further comprising applying nerve stimulation to the patient to treat bone erosion at these the first set of stimulation parameters or at a second set of stimulation parameters that are different from the first set of stimulation parameters.

10. A method of treating bone erosion in a patient, the method comprising:
identifying that the patient has failed to respond to the application of electrical energy to a nerve of an anti-inflammatory pathway in the patient to reduce inflammation; and
administering electrical energy to the nerve to treat bone erosion.

11. The method of claim 10, wherein administering electrical energy to the nerve to treat bone erosion comprises applying the same intensity of electrical energy to the nerve as was applied to reduce inflammation.

12. The method of claim 10, wherein administering electrical energy to the nerve to treat bone erosion comprises applying an intensity of electrical energy to the nerve that is less than that applied to reduce inflammation.

13. The method of claim 10, wherein administering electrical energy to the nerve to treat bone erosion comprises reducing off time between doses as compared to the application of electrical energy to a nerve of the anti-inflammatory pathway.

14. The method of claim 10, wherein the anti-inflammatory pathway in the patient comprises the patient's vagus nerve.

15. The method of claim 10, wherein the anti-inflammatory pathway in the patient comprises one or more nerve targets comprises one or more of: a cranial nerve, a peripheral nerve, a spinal nerve, or a nerve ending within an organ.

16. The method of claim 10, wherein the method further comprises administering a drug agent to treat inflammation.

17. The method of claim 16, wherein the drug agent comprises a TNF-alpha inhibitor comprising one or more of: etanercept, adalimumab, certolizumab pegol, infliximab, golimumab, and biosimilars thereof.

18. The method of claim 10, wherein the patient has an immune or inflammatory disorder.

19. The method of claim 18, wherein the immune or inflammatory disorder is one of: rheumatoid arthritis and psoriatic arthritis.

20. The method of claim 10, wherein applying electrical energy comprises applying electrical energy from an implanted device.

21. The method of claim 20, wherein the implanted device contains a pulse generator and at least one lead that terminates with at least one electrode.

22. The method of claim 21, wherein the electrode comprises one or more of: a cuff electrode, a thin film electrode, a temperature-dependent form fitting elastomer, and an in situ setting injected conductive material.

23. A method of reducing bone erosion in a patient suffering from a non-inflammatory and/or non-RANKL modulating bone erosion disorder, the method comprising administering a nerve stimulation targeting one or more of: a vagus nerve, a trigeminal nerve, a glossopharyngeal nerve, an optic nerve, or a facial nerve to reduce, eliminate or reverse bone erosion.

24. The method of claim 23, wherein the non-inflammatory bone erosion disorder comprises one or more of: osteoporosis, osteoarthritis, gout, pseudo gout, and Rheumatoid arthritis, melorheostosis and mucopolysaccharidoses.

25. The method of claim 23, wherein the nerve stimulation targets the vagus nerve.

26. The method of claim 23, wherein the nerve stimulation is applied by an implanted nerve stimulator.
